(19) United States
(12) Patent Application Publication
(10) Pub. No.: US 2010/0069274 A1
(43) Pub. Date: Mar. 18, 2010

(54) BOTTLE CONVEYOR LUBRICANT COMPOSITION AND METHOD OF USING THE SAME

(75) Inventors: Wataru Ebine, Kanagawa-ken (JP); Tsunenori Chiba, Kanagawa-ken (JP)

Correspondence Address: JohnsonDiversey, Inc., 8310 16TH STREET, M/S 509, PO BOX 902, STURTEVANT, WI, 53177-0902, US

(73) Assignee: JOHNSONDIVERSEY, INC., Sturtevant, WI (US)

(21) Appl. No.: 12/161,927

(22) PCT Filed: Jan. 24, 2007
(86) PCT No.: PCT/US2007/060988

§ 371 Date: Jul. 15, 2009

(30) Foreign Application Priority Data
Jan. 27, 2006 (JP) 2006 018477

(51) Int. Cl. C10M 137/00; C10M 145/16; C10M 129/26; A61L 2/16

(52) U.S. Cl. 508/433; 508/507; 508/459; 422/28

(57) ABSTRACT

A bottle conveyor lubricant composition used for polyalkylene terephthalate containers, characterized by comprising (A) a specific chelating compound, and (B) water, and further optionally containing (C) a nonionic surfactant, (D) a water-soluble solvent, (E) a cationic surfactant, and/or (F) an anionic surfactant. Using the specific chelating compound as a main component makes it possible to obtain a bottle conveyor lubricant suitable for moving and conveying PET containers on a stainless steel conveyor. Furthermore, by adding a specific surfactant, it is possible to obtain a bottle conveyor lubricant that can be used alone to enable PET containers to be moved and conveyed not only on stainless steel conveyors but also on resin conveyors. Moreover, the composition has excellent detergency, lubricating ability, sterilizing ability, scale suppressing ability, and stress crack preventing ability, and has an excellent effect of suppressing occurrence of deposit upon being mixed with a beverage. A method of using the composition is also provided.

### TECHNICAL FIELD

The present invention relates to a bottle conveyor lubricant that is for a bottle conveyor used in a manufacturing process or filling process for green tea, black tea, Java tea, coffee, a milk beverage, a carbonated beverage, a condiment, a processed food or the like, and is suitable in particular for moving and conveying polyalkylene terephthalate containers. More particularly, the present invention relates to a bottle conveyor lubricant that has excellent detergency, lubricity, sterilizing ability, scale suppressing ability and stress crack preventing ability, and moreover also has a deposit suppressing effect in particular.

### BACKGROUND ART

In recent years, polyalkylene terephthalate containers made of polyethylene terephthalate (PET) or the like have become widely used as beverage containers for green tea, black tea, coffee, milk beverages, carbonated beverages and the like. Furthermore, in a process of filling such a beverage into such containers, a bottle conveyor is used for moving the containers. Note that “PET container” here is a general term for ordinary PET containers that are filled with green tea, black tea, Java tea, coffee, a milk beverage or the like, and gas PET containers that are filled with a carbonated beverage or the like.

A bottle conveyor operates continuously through automatic control, and hence even if the flow of PET bottles stops, the bottle conveyor continues operating on its own, and at this time it is necessary to reduce kinetic friction between the PET bottles and the surface of the conveyor. Moreover, a suitable degree of static friction between the PET bottles and the surface of the conveyor is also required so that PET bottles that have been conveyed in from a washer can be swept along by the bottle conveyor.

Hitherto, a lubricant composition containing a surfactant having a higher fatty acid soap as a main component thereof, and further having a cationic surfactant, a nonionic surfactant or the like having a sterilizing ability blended therein as required has been disclosed in Japanese Patent Application Laid-open No. 1-96294 (Patent Document 1). This has been used by being diluted with water to a higher fatty acid concentration of approximately 0.01 to 0.1%, and then being supplied by means such as application onto a bottle conveyor.

For such a lubricant having a fatty acid soap or an alkyl phosphate ester alkali salt as a main component thereof, the lubricating performance and so on may be affected by the hardness of the water used. That is, there is a problem that such a lubricant reacts with a hardness component of the water used to produce scale, and this scale accumulates on the surface of the conveyor so that the lubricity is reduced, and moreover the appearance of microorganisms (bacteria) in the scale may be unavoidable. Moreover, there is also the problem that a lubricant supplying nozzle is clogged up by such scale. A sterilizing lubricant for moving bottles and cans comprising a specific anionic surfactant and a specific sterilizing quaternary ammonium typo cationic surfactant has thus been disclosed in Japanese Patent Application Laid-open No. 2-97592 (Patent Document 2).

Furthermore, there is a problem that upon such a lubricant becoming attached to a PET bottle for a carbonated beverage, because the inside of the bottle is in a pressurized state due to carbon dioxide gas, the PET bottle is subjected to stress so that stress cracks appear, and thus the risk of the PET bottle failing, and the risk of the beverage filled inside the bottle leaking are unavoidable; in Japanese Patent Application Laid-open No. 6-172773, there is thus disclosed a bottle conveyor lubricant for PET containers in a process of bottling a carbonated beverage, in which a lubricant composition having a water-soluble fatty acid alkali salt as a main component thereof is made to further contain an alkyl diphenyl ether disulfonate, whereby stress cracking of the PET containers can be prevented or suppressed while maintaining lubricity (Patent Document 3) .

As the material of a bottle conveyor used for moving and conveying PET containers, a resin such as a polyacetal resin, a polypropylene resin, a polyethylene resin, or an acrylonitrile-butadiene-styrene resin is used. From hitherto, PET container lubricants have thus been designed presupposing use with a resin bottle conveyor.

As a lubricant for a resin conveyor, in Japanese Patent Application Laid-open No. 10-158681, there is disclosed a lubricant for a bottle conveyor made of a synthetic resin such as a polyacetal resin that, which has as a main component thereof an aqueous solution of not less than 0.0025 wt % of a polyethylene glycol nonionic surfactant consisting of at least one selected from polyoxyethylene alkyl ethers, polyoxyethylene fatty acid esters, and block copolymers between polyoxyethylene and polyoxypropylene, whereby detergency and lubricity are excellent, and scale is not produced (Patent Document 4).

However, for the above lubricant, with a conveyor made of a metal such as stainless steel used in a process with a shrink labeler or an inverting sterilizer (hereinafter referred to as a “stainless stool conveyor”), the desired lubricating performance cannot obtained, and hence it has not been possible to use such a lubricant for conveying PET containers. Moreover, there would be a risk of problems being caused with regard to the manufacturing efficiency, and hence for stainless steel conveyors in such processes, a separate lubricant for conveying PET containers on stainless steel conveyors has been used.

Moreover, for a lubricant having a fatty acid soap or an alkyl phosphate ester alkali salt as a main component thereof as described above, the lubricating performance and so on are affected by the hardness of the water used, and hence there are a problem that scale accumulates on the surface of the conveyor so that the lubricity is reduced, and a problem that microorganisms (bacteria) appear in the scale. Various improvements have been proposed to combat this, but the cost has tended to increase.

Moreover, in Japanese Patent Application Laid-open No. 2000-129277, there is disclosed a sterilizing lubricant for conveyors, which is a lubricant used by being applied or the like to a container-conveying conveyor when conveying containers such as cans or bottles into which is filled a beverage such as green tea, black tea, coffee, cola or beer on the conveyor, and contains an alkyl pyridinua chloride as a main component thereof, whereby the sterilizing ability is excellent, and moreover deposit is not produced upon contact with the beverage on the conveyor, and hence a reduction in lubricity or soiling due to such deposit on the conveyor is not brought about, excellent lubricity always being maintained (Patent Document 5). However, there has been a problem that to form this sterilizing lubricant for conveyors, the choice of the sterilizing component is limited to an alkyl pyridinium chloride. Moreover, with increases in demand for and production of green tea in recent years, compatibility with beverage components has come to be required, it being strongly required to prevent soiling of a conveyor, the surroundings thereof, and containers on the conveyor due to deposit, and moreover prevent a decrease in lubricity due to such deposit.

### DISCLOSURE OF THE INVENTION

The present invention has been devised in view of the state of affairs described above, and provides a lubricant for use with stainless steel conveyors suitable for moving and conveying PET containers. Moreover, it is an object to provide a bottle conveyor lubricant that can be used alone to enable PET containers to be moved and conveyed not only on stainless steel conveyors but also on resin conveyors. Furthermore, an object is to provide a bottle conveyor lubricant suitable for moving and conveying PET containers, according to which detergency, lubricity, and sterilizing ability are excellent, and moreover production of scale and stress cracking are prevented. Note that “resin conveyors” here means conveyors made of a resin material such as a polyacetal resin, a polypropylene resin, a polyethylene resin, or an acrylonitrile-butadiene-styrene resin.

Moreover, there is a problem that in a filling process or the like for a beverage such as green tea, black tea, Java tea, coffee, beer or cola, particularly green tea or black tea, upon the beverage overflowing and becoming attached, components of the beverage and components of the conveyor lubricant form a water-insoluble salt (deposit) through chemical reaction, which becomes attached as a brown-black deposit to the conveyor and also to the bottoms and sides of PET containers.

The present inventors carried out assiduous studies to attain the above objects, and as a result, by using a specific chelating compound as a main component, have been able to obtain a bottle conveyor lubricant suitable for moving and conveying PET containers on a stainless steel conveyor.

Moreover, by adding a specific surfactant to the specific chelating compound, a bottle conveyor lubricant can be obtained that enables PET containers to be moved and conveyed not only on stainless steel conveyors but also on resin conveyors. Furthermore, it has been discovered that such a bottle conveyor lubricant has excellent detergency, lubricity, and sterilizing ability, and moreover has an excellent effect of preventing scale production and stress cracking, thus accomplishing the present invention.

Moreover, such a bottle conveyor lubricant has excellent compatibility with the components of beverages such as green tea, black tea, Java tea, coffee, beer and cola, particularly green tea and black tea, and hence in a filling process or the like for such a beverage, there can be resolved the problem of, upon the beverage overflowing and becoming attached, components of the beverage and components of the conveyor lubricant forming a water-insoluble salt (deposit) through chemical reaction, which becomes attached as a brown-black deposit to the conveyor and also to the bottoms and sides of PET containers.

That is, a first aspect of the present invention is a bottle conveyor lubricant composition characterized by containing (A) at least one chelating compound selected from the group consisting of phosphonic acids, polyacrylic acid polymers and/or salts thereof (wherein the salts comprise alkali metal salts, alkaline earth metal salts, ammonium salts, or amine salts), and (B) water.

Furthermore, a second aspect is the above bottle conveyor lubricant composition further containing (C) a nonionic surfactant, a third aspect is the above bottle conveyor lubricant composition further containing (D) a water-soluble solvent, and a fourth aspect is the above bottle conveyor lubricant composition further containing (E) a cationic surfactant.

Moreover, a fifth aspect is the above bottle conveyor lubricant composition further containing (F) at least one anionic surfactant selected from the group represented by general formulae (1) to (3)

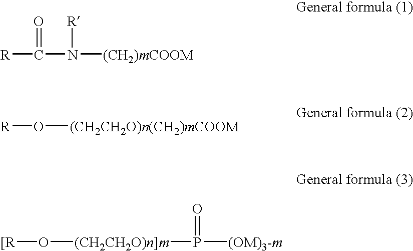

wherein R is a C₃-C₂₀ alkyl group or alkenyl group, R′ is a C₁-C₄ alkyl group, n is an integer from 1 to 8, m is an integer of 1 or 2, and M is hydrogen, an alkali metal, an amine, or an alkanolamine.

Moreover, a sixth aspect is the above bottle conveyor lubricant composition, wherein the nonionic surfactant component (C) comprises at least one selected from polyoxyalkylene alkyl ethers, polyoxyalkylene fatty acid esters, block copolymers of polyoxyethylene (EO) and polyoxypropylene (PO), and polyoxyalkylene styrenated phenyl ethers.

Moreover, a seventh aspect is the above bottle conveyor lubricant composition, wherein the water-soluble solvent component (D) comprises at least one selected from the group consisting of ethyl alcohol, isopropyl alcohol, propylene glycol, polyethylene glycol, ethylene glycol ethers including ethylene glycol monomethyl ether, ethylene glycol monoethyl ether, ethylene glycol monopropyl ether and ethylene glycol monobutyl ether, diethylene glycol ethers including diethylene glycol monomethyl ether, diethylene glycol monoethyl ether, diethylene glycol monopropyl ether and diethylene glycol monobutyl ether, propylene glycol monomethyl ether, dipropylene glycol monomethyl ether, hexylene glycol, isoprene glycol, and glycerol.

Moreover, an eighth aspect is the bottle conveyor lubricant composition according to anyone of the above first to seventh aspects, the bottle conveyor lubricant composition being used for conveying polyalkylene terephthalate containers, and a ninth aspect is the bottle conveyor lubricant composition according to anyone of the above first to eighth aspects, the bottle conveyor lubricant composition having an effect of suppressing the occurrence of deposit upon contact with a beverage to be filled on a conveyor.

Furthermore, a tenth aspect is a method of use comprising using the bottle conveyor lubricant composition according to any one of the above first to ninth aspects for conveying and moving containers on a conveyor by supplying, spraying, or applying onto the conveyor a diluted lubricant liquid obtained by diluting the lubricant composition with water or hot water to a component (A) concentration of 10 to 500 mg/L.

Moreover, an eleventh aspect is a method of use comprising using the bottle conveyor lubricant composition according to any one of the above first to ninth aspects for conveying and moving containers by supplying, spraying, or applying onto a conveyor a mixture of a diluted lubricant liquid obtained by diluting the lubricant composition with water or hot water to a component (A) concentration of 10 to 500 mg/L, and at least one selected from sterilizing agent aqueous solutions obtained by diluting, with water or hot water, a hypochlorite, peracetic acid, hydrogen peroxide or iodine to not less than 5 mg/L or chlorine dioxide to not less than 0.05 mg/L, and a twelfth aspect is a method of use comprising using the bottle conveyor lubricant composition according to any one of the above first to ninth aspects for conveying and moving containers by separately supplying, spraying, or applying onto a conveyor a diluted lubricant liquid obtained by diluting the lubricant composition with water or hot water to a component (A) concentration of 10 to 500 mg/L, and at least one selected from sterilizing agent aqueous solutions obtained by diluting, with water or hot water, a hypochlorite, peracetic acid, hydrogen peroxide or iodine to not less than 5 mg/L or chlorine dioxide to not less than 0.05 mg/L.

According to the present invention, by using a specific chelating compound as a main component, a bottle conveyor lubricant suitable for moving and conveying PET containers on a stainless steel conveyor can be obtained.

Moreover, by adding a specific surfactant to the specific chelating compound, a bottle conveyor lubricant can be obtained that enables PET containers to be moved and conveyed not only on stainless steel conveyors but also on resin conveyors. That is, the bottle conveyor lubricant containing the specific chelating compound and the specific surfactant has the advantage of being usable not only on stainless steel conveyors used in processes with shrink labelers and inverting sterilizers, but also on resin conveyors used in other processes. Furthermore, the bottle conveyor lubricant has the advantage of having excellent detergency, lubricity, sterilizing ability, scale suppressing ability and stress crack preventing ability, and moreover an excellent deposit suppressing effect in particular.

In particular, for the bottle conveyor lubricant composition of the present invention, due it being possible to use a chelating compound as the main component thereof, good detergency not achieved conventionally can be secured, and hence there is the advantage that the problem of the lubricating performance and so on being affected by the hardness of the water used, the problem of accumulation on the surface of the conveyor so that the lubricity is reduced, and the problem of microorganisms (bacteria) appearing in the accumulated scale can be resolved inexpensively.

Furthermore, the bottle conveyor lubricant has excellent compatibility with the components of beverages such as green tea, black tea, Java tea, coffee, beer and cola, particularly green tea and black tea, and hence in a filling process or the like for such a beverage, there is the advantage that, upon the beverage overflowing and becoming attached, components of the beverage and components of the conveyor lubricant do not form a water-insoluble salt (deposit) through chemical reaction, and thus such a deposit does not become attached as a brown-black deposit to the conveyor and also to the bottoms and sides of PET containers.

In addition, the lubricant composition of the present invention is highly compatible with cationic surfactants and other sterilizing agents, and hence has the advantage that an equivalent or better sterilizing ability can be obtained with a lower amount of a cationic surfactant or other sterilizing agent blended in than for a conventional sterilizing lubricant composition.

### BEST MODE FOR CARRYING OUT THE INVENTION

Next, a best mode for carrying out the present invention will be described in detail.

The present invention relates to a bottle conveyor lubricant composition (hereinafter sometimes referred to as the “lubricant composition”) characterized by containing (A) a specific chelating compound, and (B) water, and further optionally containing (C) a nonionic surfactant, (D) a water-soluble solvent, (E) a cationic surfactant, and/or (F) an anionic surfactant, and a method of using the lubricant composition.

The chelating compound comprising component (A) used in the lubricant composition of the present invention comprises at least one chelating compound selected from the group consisting of phosphonic acids and polyacrylic acid polymers and/or salts thereof (wherein the salts comprise alkali metal salts, alkaline earth metal salts, ammonium salts, and amine salts).

More specifically, examples of phosphonic acids and/or salts thereof include 2-phosphono-1,2,4-butanetricarboxylic acid, methyl diphosphonic acid, aminotrismethylene phosphonic acid, ethylidene diphosphonic acid, 1-hydroxyethylidene-1,1-diphosphonic acid, 1-hydroxypropylidene-1,1-diphosphonic acid, ethyl amino bismethylene phosphonic acid, dodecyl amino bismethylene phosphonic acid, nitrilo trismethylene phosphonic acid, ethylene diamine bismethylene phosphonic acid, ethylene diamine tetrakismethylene phosphonic acid, hexene diamine tetrakismethylene phosphonic acid, diethylene triamine pentamethylene phosphonic acid, and 1,2-propanediamine tetramethylene phosphonic acid, and also ammonium salts, alkali metal salts, and organic amine salts thereof and derivatives thereof such as oxidized derivatives in which a nitrogen atom in the molecule is oxidized to form an N-oxide.

Moreover, examples of polyacrylic acid polymers and/or salts thereof include polyacrylic acid polymers and/or salts thereof such as polyacrylic acid polymers, acrylic acid/methacrylic acid copolymers, acrylic acid/methacrylic acid ester copolymers, acrylic acid/maleic acid copolymers, methacrylic acid/maleic acid copolymers, and isoamylene/maleic anhydride copolymers, and also ammonium salts, alkali metal salts, and organic amine salts thereof.

Such a chelating compound comprising component (A) may be used alone, or a plurality may be used in combination. The proportion of component (A) in the lubricant composition is set in a range of 0.2 to 20 mass %. That is, it is undesirable for this proportion to be less than 0.2 mass % in terms of manufacturing/distribution/storage costs and so on from the viewpoint of making the lubricant composition compact; moreover, if this proportion is greater than 20 mass %, then there will be no further improvement in lubricating performance, and moreover this is undesirable economically.

Examples of the water comprising component (B) used in the lubricant composition of the present invention include pure water, ion exchange water, soft water, distilled water, and tap water. One of these may be used alone, or a plurality may be used in combination. Of the above, from the viewpoint of economics and storage stability, tap water or ion exchange water is preferably used. Note that the “water” is the total of water contained in the form of aqueous solutions or waters of crystallization originating from components of the lubricant composition of the present invention, and other water added from outside, the blending being carried out such that the composition as a whole becomes 100%.

By using an aqueous solution of the specific chelating compound described above and/or a diluted liquid thereof, a desired lubricating performance can be obtained on a stainless steel conveyor for enabling polyalkylene terephthalate containers to be moved and conveyed.

Examples of the nonionic surfactant comprising component (C) used in the lubricant composition of the present invention are (1) polyoxyalkylene alkyl ethers, (2) polyoxyalkylene fatty acid esters, (3) block copolymers of polyoxyethylene (EO) and polyoxypropylene (PO), and (4) polyoxyalkylene styrenated phenyl ethers. By blending in such a specific nonionic surfactant, the desired lubricating performance can be obtained such that polyalkylene terephthalate containers can be moved and conveyed not only on stainless steel conveyors but also on plastic conveyors.

A polyoxyalkylene alkyl ether of (1) above is a compound having an HLB of 9 to 21 obtained by adding polyoxyethylene (EO), polyoxypropylene (PO) or polyoxybutylene (BO) to a primary or secondary alcohol having 10 to 21 carbon atoms, preferably a compound having an HLB of 12 to 21 obtained by adding polyoxyethylene to a primary alcohol.

Specific examples of such compounds include a polyoxyethylene isodecyl ethoxylate having an HLB of 19.3 obtained by adding polyoxyethylene (EO 100 mol) to isodecyl alcohol which has 10 carbon atoms, a compound having an HLB of 13.0 obtained by adding polyoxyethylene (EO 9 mol) to a mixed alcohol comprising tridecyl alcohol which has 13 carbon atoms and myristyl alcohol which has 14 carbon atoms, a polyoxyethylene myristyl ethoxylate having an HLB of 13.0 obtained by adding polyoxyethylene (EO 9 mol) to myristyl alcohol which has 14 carbon atoms, a polyoxyethylene pentadecyl ethoxylate having an HLB of 13.0 obtained by adding polyoxyethylene (EO 10 mol) to pentadecyl alcohol which has 15 carbon atoms, a polyoxyethylene cetyl ethoxylate having an HLB of 14.2 obtained by adding polyoxyethylene (EO 13 mol) to cetyl alcohol which has 16 carbon atoms, a polyoxyethylene pentadecyl ethoxylate having an HLB of 13.0 obtained by adding polyoxyethylene (EO 11 mol) to heptadecyl alcohol which has 17 carbon atoms, a polyoxyethylene stearyl ethoxylate having an HLB of 13.9 obtained by adding polyoxyethylene (EO 13 mol) to stearyl alcohol which has 18 carbon atoms (saturated), a polyoxyethylene oleyl ethoxylate having an HLB of 16.6 obtained by adding polyoxyethylene (EO 30 mol) to oleyl alcohol which has 18 carbon atoms (unsaturated), and compounds having an HLB of 9 to 19 obtained by adding polyoxyethylene (EO 5 to 35) to a secondary alcohol having 10 to 18 carbon atoms.

A polyoxyalkylene fatty acid ester of (2) above is a polyoxyethylene mono- or di-fatty acid ester, preferably a polyoxyethylene mono-fatty acid ester, the fatty acid having 10 to 18 carbon atoms, and the compound having an HLB of 9 to 19, preferably 12 to 19.

Specific examples of such compounds include a polyoxyethylene monolaurate (HLB 9.5), a polyoxyethylene monolaurate (HUB 13.1), a polyoxyethylene monooleate (HLB 11.5), a polyoxyethylene monooleate (HLB 13.5), a polyoxyethylene dioleate (HLB 10.4), a polyoxyethylene monostearate (HLB 18), and a polyoxyethylene distearate (HLB 18.6).

A block copolymer of polyoxyethylene (EO) and polyoxypropylene (PO) of (3) above is a compound having an EC) content of 70 to 80% and a molecular weight of 2,000 to 3,500, specific examples including Pluronic F-88 (EO content 80%, molecular weight 2,250) and Pluronic F-108 (EO content 80%, molecular weight 3,250) (both made by Adeka Corporation).

Examples of polyoxyalkylene styrenated phenyl ethers of (4) above include Noigen EA207D (HLB 18.7, made by Dal-ichi Kogyo Seiyaku Co., Ltd.).

Such a nonionic surfactant comprising component (C) in the present invention may be used alone, or a plurality may be used in combination. The proportion of the nonionic surfactant in the lubricant composition is set in a range of 0.2 to 20 mass %. That is, it is undesirable for this proportion to be less than 0.2 mass % in terms of manufacturing/distribution/storage costs and so on from the viewpoint of making the lubricant composition compact; moreover, if more than 20 mass % is blended in, there will be no further improvement in lubricating performance, and moreover this is undesirable economically.

Moreover, the mass ratio between component (A) and component (C) in the lubricant composition is preferably in a range of A:C=1:10 to 20:1, and the content of component (A) plus component (C) in the lubricant composition is preferably in a range of 1 to 40 mass %.

If the A:C mass ratio is less than 1:10, then from the balance with the other components, the low foaming property and storage stability will be poor, whereas if the A:C mass ratio is greater than 20:1, then from the balance with the other components, the storage stability will again be poor. Moreover, if the content of component (A) plus component (C) is less than 1 mass %, then the desired lubricating performance will not be obtained, whereas if more than 40 mass % is blended in, there will be no further improvement in lubricating performance, and moreover this is undesirable economically.

Examples of the water-soluble solvent comprising component (D) used in the present invention are alcohols such as ethyl alcohol and isopropyl alcohol, glycols such as propylene glycol, polyethylene glycol, hexylene glycol and isoprene glycol, ethylene glycol ethers such as ethylene glycol monomethyl ether, ethylene glycol monoethyl ether, ethylene glycol monopropyl ether and ethylene glycol monobutyl ether, diethylene glycol ethers such as diethylene glycol monomethyl ether, diethylene glycol monoethyl ether, diethylene glycol monopropyl ether and diethylene glycol monobutyl ether, propylene glycol ethers such as propylene glycol monomethyl ether and dipropylene glycol monomethyl ether, amines such as monoethanolamine, diethanolamine, triethanolamine and isopropanolamine, glycerol, and polyethylene glycol. Of these, propylene glycol, hexylene glycol, isoprene glycol, ethyl alcohol, and isopropyl alcohol are preferably used.

Such a water-soluble solvent comprising component (D) in the present invention may be used alone, or a plurality may be used in combination. The proportion of component (D) in the lubricant composition is set in a range of 0.8 to 38 mass %. That is, if this proportion is less than 0.8 mass %, then good storage stability (0° C.) will not be obtained for the lubricant composition, whereas if more than 38 mass % is blended in, there will be no further improvement in the storage stability, and moreover this is undesirable economically from a cost perspective.

The cationic surfactant comprising component (E) used in the present invention is preferably any of various quaternary ammonium salts, specific examples including dimethyl benzyl ammonium chloride, didecyl dimethyl ammonium chloride, didecyl dimethyl ammonium adipate, didecyl dimethyl ammonium gluconate, didecyl monomethyl hydroxyethyl ammonium chloride, didecyl monomethyl hydroxyethyl ammonium adipate, didecyl monomethyl hydroxyethyl ammonium gluconate, didecyl monomethyl hydroxyethyl ammonium sulfonate, didecyl dimethyl ammonium propionate, and hexadecyl tributyl phosphonium. From the viewpoint to the strength of the sterilizing power in particular, the number of carbon atoms in an alkyl group is set in a range of 10 to 16. Moreover, examples of biguanide type cationic surfactants include polyhexamethylene biguanide hydrochloride. One of these may be used alone, or a plurality may be used in combination. Of the above, an alkyl dimethyl hydroxyethyl ammonium chloride, dimethyl benzyl ammonium chloride, polyhexamethylene biguanide hydrochloride, didecyl dimethyl ammonium chloride, didecyl dimethyl ammonium adipate, and hexadecyl tributyl phosphonium are preferably used.

Such a cationic surfactant comprising component (E) in the present invention may be used alone, or a plurality may be used in combination. The proportion of component (E) in the lubricant composition is set in a range of 0.08 to 30 mass % in terms of the amount of the active constituent. That is, if this proportion is less than 0.08 mass %, then the sterilizing performance will be poor, whereas if more than 30 mass % is blended in, from the balance with the other components, the desired storage stability will not be obtained, and moreover this is undesirable economically from a cost perspective. Note that to effectively achieve the sterilizing performance, dilution/adjustment is preferably carried out such that the cationic surfactant concentration in the diluted lubricant liquid is in a range of 5 to 1200 mg/L. Moreover, for a lubricant composition not containing an anionic surfactant, by carrying out dilution/adjustment to a cationic surfactant concentration in a range of 5 to 800 mg/L, the desired sterilizing performance can be achieved.

Examples of the anionic surfactant comprising component (F) used in the present invention are ones represented by general formulae (1) to (3).

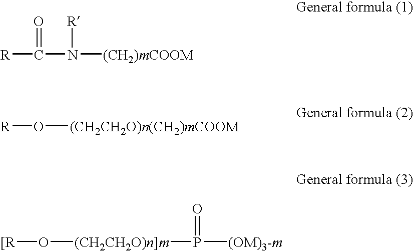

Here, R is a C₃-C₂₀ alkyl group or alkenyl group. R′ is a C₁-C₄ alkyl group. n is an integer from 1 to 8. m is an integer of 1 or 2. M is hydrogen, an alkali metal, an amine, or an alkanolamine.

More specifically, examples of anionic surfactants of general formula (1) are oleoyl sarcosine, capryloyl sarcosine, N-oleoyl-N-butylglycine, oleyl alanine, and linoloyl sarcosine, and alkali metal salts, amine salts and alkanolamine salts thereof; examples of anionic surfactants of general formula (2) are polyoxyethylene oleyl ether acetate sodium salt, polyoxyethylene capryl ether acetate triethanolamine salt, polyoxyethylene lauryl ether acetic acid, polyoxyethylene lauryl ether acetate sodium salt, polyoxyethylene lauryl ether propionate potassium salt, and polyoxyethylene eicosyl ether acetate sodium salt; examples of anionic surfactants of general formula (3) include polyoxyethylene stearyl ether phosphate triethanolamine salt, polyoxyethylene capryl ether phosphate morpholine, polyoxyethylene lauryl ether phosphate sodium salt, and polyoxyethylene lauryl ether phosphate amine salts. Of these, polyoxyethylene lauryl ether phosphate sodium salt, polyoxyethylene capryl ether phosphate monoethanolamine salt, polyoxyethylene oleyl ether acetate sodium salt, and polyoxyethylene oleyl ether acetate monoethanolamine salt are preferably used.

Such an anionic surfactant comprising component (F) in the present invention may be used alone, or a plurality may be used in combination. The proportion of component (F) in the lubricant composition is set in a range of 0.2 to 20 mass % in terms of the amount of the active constituent. That is, if this proportion is less than 0.2 mass %, then the storage stability due to improved solubilization will be poor, whereas if more than 20 mass % is blended in, from the balance with the other components, there will be no further improvement in the storage stability due to improved solubilization, and moreover this is undesirable from a cost perspective.

The lubricant composition of the present invention may further contain other optional components such as sterilizing agents, detergent builders, amphoteric surfactants, antifoaming agents, and cloud point improvers as required.

Of such optional components, examples of detergent builders are carboxylic acids and/or salts thereof, for example ethylenediaminetetraacetic acid, nitrilotriacetic acid, iminodiacetic acid, citric acid, malic acid, tartaric acid, succinic acid, gluconic acid and fumaric acid, and carboxylates thereof such as alkali metal salts and ammonium salts.

The bottle conveyor lubricant composition of the present invention is used for conveying and moving PET containers as a diluted lubricant liquid obtained by diluting to a concentration of the chelating compound comprising component (A) of 10 to 500 mg/L. The dilution of the lubricant is generally carried out using water; the lubricant may be used diluted to the desired usage concentration at the time of use, or may be diluted to a suitable concentration to produce a solution in advance and then used by further diluting this solution to the desired usage concentration at the Lime of use. The diluted lubricant liquid thus obtained may be sprayed onto a conveyor continuously or intermittently from a nozzle via a pump or the like, or applied onto the conveyor with a brush or the like.

At this time, the concentration of the nonionic surfactant is preferably not less than 25 mg/L, particularly preferable in a range of 25 to 1,000 mg/L, in the diluted lubricant liquid obtained through the dilution. If this content is less than 25 mg/L, then a decrease in lubricity, detergency and so on will be brought about, whereas if this content is greater than 1,000 mg/L, then although the lubricity and detergency will be further increased, drawbacks will arise such as the cost increasing.

Furthermore, for the bottle conveyor lubricant composition of the present invention, a method may be adopted in which the above diluted lubricant liquid is mixed with at least one selected from sterilizing agent aqueous solutions obtained by diluting, with water or hot water, a hypochlorite, peracetic acid, hydrogen peroxide or iodine to not less than 5 mg/L or chlorine dioxide to not less than 0.05 mg/L, and the mixture is supplied, sprayed, or applied onto the conveyor.

Alternatively, a method may be adopted in which the above diluted lubricant liquid, and at least one selected from sterilizing agent aqueous solutions obtained by diluting, with water or hot water, a hypochlorite, peracetic acid, hydrogen peroxide or iodine to not less than 5 mg/L or chlorine dioxide to not less than 0.05 mg/L, are supplied or sprayed onto the conveyor continuously or intermittently via separately provided nozzles, or applied onto the conveyor.

As described above, the lubricant composition of the present invention is made to have a specific chelating compound as a main component thereof, whereby a bottle conveyor lubricant suitable for moving and conveying PET containers on a stainless steel conveyor can be obtained.

Moreover, by adding a specific surfactant to the specific chelating compound, a bottle conveyor lubricant can be obtained that enables PET containers to be moved and conveyed not only on stainless steel conveyors but also on resin conveyors. That is, the bottle conveyor lubricant containing the specific chelating compound and the specific surfactant achieves the effect of being usable not only on stainless steel conveyors used in processes with shrink labelers and inverting sterilizers, but also on resin conveyors used in other processes. Furthermore, the bottle conveyor lubricant has the effect of having excellent detergency, lubricity, sterilizing ability, scale suppressing ability and stress crack preventing ability, and moreover a particularly excellent deposit suppressing effect.

### EXAMPLES

Next, examples will be described together with comparative examples.

Test lubricant compositions of compositions for examples 1 to 56 and comparative examples 1 to 6 as shown in Tables 1 to 15 below (units of numerical values in each of the tables are mass %) were prepared, and the six items lubricity, detergency, storage stability, scale suppressing ability, deposit suppressing ability, and stress crack preventing ability were evaluated, and moreover for examples 15, 16, and 33 to 56, and comparative examples 1 to 6, the item sterilizing ability was also evaluated. Note that the amounts blended in of the respective components in the tables are shown as is, and the amounts of the active constituents in the components (mass %) are as below. Moreover, the test results are also shown in Tables 1 to 15 below. The test method and evaluation criteria for each of the items are as indicated below.

### Lubricity Test 1

### Test Method

Test PET containers were placed on a stainless steel conveyor plate, and then a test diluted lubricant liquid obtained by diluting one of the test lubricant compositions with water to a concentration of the chelating compound comprising component (A) of 25 mg/L was supplied onto the conveyor plate at 25 ml/min and the coefficient of friction (μ) after 10 minutes was measured, so as to evaluate the lubricity for each of the test lubricant compositions. Note that for the comparative examples which did not contain the chelating compound comprising component (A), each test diluted lubricant liquid used in the test was obtained by diluting with water by a factor of 400. Moreover, the operating speed of the conveyor was made to be 40 cm/s, and two test PET containers (total weight 1070 g) were used as the test bottles. The coefficient of friction (μ) was calculated using the following calculation formula, and judgment was carried out in accordance with the following evaluation criteria.

Coefficient of friction (μ)=tensile resistance according to spring balance (g)/weight of test bottles (g) (Formula 1)

### Evaluation Criteria

◯: Coefficient of friction (μ) less than 0.2
x: Coefficient of friction (μ) not less than 0.2

The case that the value of the coefficient of friction (μ) obtained was less than 0.2 was judged as “lubricity good”, whereas the case that this value was not less than 0.2 was judged as “lubricity poor”.

### Lubricity Test 2

### Test Method

Test PET containers were placed on a polyacetal resin conveyor plate, and then a diluted lubricant liquid obtained by diluting one of the test lubricant compositions with water to a concentration of the nonionic surfactant comprising component (C) of 25 mg/L was supplied onto the conveyor plate at 25 ml/min and the coefficient of friction (μ) after 10 minutes was measured, so as to evaluate the lubricity for each of the test lubricant compositions. Note that for the comparative examples which did not contain the nonionic surfactant comprising component (C), each test diluted lubricant liquid used in the test was obtained by diluting with water by a factor of 400. Moreover, the operating speed of the conveyor was made to be 40 cm/s, and two test PET containers (total weight 1070 g) were used as the test bottles. The coefficient of friction (μ) was calculated using the following calculation formula, and judgment was carried out in accordance with the following evaluation criteria.

Coefficient of friction (μ)=tensile resistance according to spring balance (g)/weight of test bottles (g) (Formula 2)

### Evaluation Criteria

◯: Coefficient of friction (μ) less than 0.1
x: Coefficient of friction (μ) not less than 0.1

The case that the value of the coefficient of friction (μ) obtained was less than 0.1 was judged as “lubricity good”, whereas the case that this value was not less than 0.1 was judged as “lubricity poor”.

### Lubricity Test 3

### Test Method

500 ml capacity carbonated beverage PET containers were each filled with carbonated water, and the pressure was adjusted to 4.0 to 4.5 gas vol (a pressure of carbon dioxide gas 4.0 to 4.5 times the capacity of the container), thus preparing test gas PET containers. The test gas PET containers were placed on a polyacetal resin conveyor plate, and then a diluted lubricant liquid obtained by diluting one of the test lubricant compositions with water to a concentration of the nonionic surfactant comprising component (C) of 25 mg/L was supplied onto the conveyor plate at 25 ml/min and the coefficient of friction (μ) after 10 minutes was measured, so as to evaluate the lubricity for each of the test lubricant compositions. Note that for the comparative examples which did not contain the nonionic surfactant comprising component (C), each test diluted lubricant liquid used in the test was obtained by diluting with water by a factor of 400. Moreover, the operating speed of the conveyor was made to be 40 cm/s, and two test gas PET containers (total weight 1070 g) were used as the test bottles The coefficient of friction (μ) was calculated using the following calculation formula, and judgment was carried out in accordance with the following evaluation criteria.

Coefficient of friction (μ)=tensile resistance according to spring balance (g)/weight of test bottles (g) (Formula 3)

### Evaluation Criteria

◯: Coefficient of friction (μ) less than 0.1
x: Coefficient of friction (μ) not less than 0.1

The case that the value of the coefficient of friction (μ) obtained was less than 0.1 was judged as “lubricity good”, whereas the case that this value was not less than 0.1 was judged as “lubricity poor”.

### Deposit Suppression Test

### Test Method

(1) An aqueous solution obtained by diluting each test lubricant composition with tap water by a factor of 400, and a beverage (green tea or black tea) were mixed together in a ratio of aqueous solution:beverage=5:1, the mixture was left for 3 hours in a water bath at 80° C., it was visually inspected whether deposit was produced, and judgment was carried out in accordance with the following evaluation criteria. Moreover, in the case of beer, the aqueous solution and the beer were mixed together in a ratio of aqueous solution:beer=1:1, the mixture was left for 3 hours in a water bath at 4° C., it was visually inspected whether deposit was produced, and judgment was again carried out in accordance with the following evaluation criteria.

(2) Moreover, in addition, an aqueous solution obtained by diluting each lubricant composition by a factor of 400 and green tea were mixed together in a ratio of 5:1 on a conveyor, it was inspected whether or not deposit was produced on the conveyor at this time, and judgment was carried out in accordance with the following evaluation criteria.

### Evaluation Criteria

◯: No deposit seen at all
Δ: Small amount of deposit seen (no problem in practice)
x: Marked production of deposit seen
◯ and Δ in the evaluation criteria were judged as meaning usable in practice.

### Detergency Test

### Test Method

The state of black soiling of the surface of the conveyor after the completion of the above lubricity test was visually inspected, and judgment was carried out in accordance with the following evaluation criteria.

### Evaluation Criteria

◯: No soiling seen, conveyor plate uniformly wet
Δ: No soiling seen, conveyor plate almost uniformly wet. (no problem in practice)
x: Soiling seen
◯ and Δ in the evaluation criteria were judged as meaning usable in practice.

### Scale Suppression Test

### Test Method

For 200 ml of a test diluted lubricant liquid obtained by diluting each of the test lubricant compositions to a concentration of the chelating compound comprising component (A) of 25 mg/L using hard water to which calcium carbonate had been added to a concentration of 100 mg/L, the appearance after 1 hour (room temperature) was visually inspected, and judgment was carried out in accordance with the following evaluation criteria. Note that for the comparative examples which did not contain the chelating compound comprising component (A), each test diluted lubricant liquid used in the test was obtained by diluting with the water by a factor of 400.

### Evaluation Criteria

◯: Transparent, no cloudiness, no precipitation of deposit
Δ: Transparent to slightly cloudy, no precipitation of deposit (no problem in practice)
x: Cloudy, deposit precipitates out
◯ and Δ in the evaluation criteria were judged as meaning usable in practice.

### Storage Stability Test

### Test Method

500 ml of each of the test lubricant compositions was sealed in a 500 ml capacity glass container, and was then left for 10 days either at room temperature or in an incubator (model Bitec 500, made by Shimadzu Corporation) set to 0° C., and judgment was carried out visually in accordance with the following evaluation criteria.

### Evaluation Criteria

◯: Uniformly transparent at both room temperature and 0° C., no solidification or separation seen
Δ: Uniformly transparent at both room temperature and 0° C., but slight solidification or separation seen at 0° C.
x: Becomes turbid (not transparent) at both room temperature and 0° C., solidification or separation seen
◯ and Δ in the evaluation criteria were judged as meaning usable in practice.

### Stress Cracking Test

### Test Method

A PET container was filled with carbonated water, and the pressure was adjusted to 4.0 to 4.5 gas vol (a pressure of carbon dioxide gas 4.0 to 4.5 times the capacity of the container), thus preparing a test PET container. Next, each test lubricant composition in Table 1 was diluted with water to a concentration of the chelating compound comprising component (A) of 25 mg/L so as to prepare a test diluted lubricant liquid, the test PET container was semi-immersed therein, and left for 10 days under conditions of 40° C. and 80% humidity, and then the state of cracking of the PET container was visually inspected, and judgment was carried out in accordance with the following evaluation criteria. Note that for the comparative examples which did not contain the chelating compound comprising component (A), each test diluted lubricant liquid used in the test was obtained by diluting with water by a factor of 400.

### Evaluation Criteria

A: No cracking at all or hardly any cracking
B: Some small cracks, but hardly any
C: Large cracks seen
D: Many large cracks
A and B in the evaluation criteria were judged as meaning usable in practice

### Sterilizing Ability Test

### Test Method

Following European Standard test method EN 1040, coli bacteria or Pseudomonas aeruginosa was brought into contact for 15 minutes with a diluted lubricant liquid obtained by diluting one of the test lubricant compositions with pure water to a concentration of the nonionic surfactant comprising component (C) of 25 mg/L, application onto an agar culture medium was carried out and culturing was carried out for 48 hours at 37° C., and judgment was carried out in accordance with the following evaluation criteria from the logarithmic decrease in the number of bacteria. Note that for examples 15 and 16 and the comparative examples which did not contain the nonionic surfactant comprising component (C), each test diluted lubricant liquid used in the test was obtained by diluting with pure water by a factor of 400.

### Evaluation Criteria

◯: Logarithmic decrease from initial number of bacteria not less than 5 log (good sterilizing ability)
x: Logarithmic decrease from initial number of bacteria less than 5 log (poor sterilizing ability)

### Component A

### Phosphonic Acid 1

1-hydroxyethylidene-1,1-diphosphonic acid
Trade name: “Chelest PH-210”, made by Chelest Corporation (active constituent 60 mass %)

### Phosphonic Acid 2

2-phosphono-1,2,4-butanetricarboxylic acid
Trade name: “Chelest PH-430”, made by Chelest Corporation (active constituent 50 mass %)

### Phosphonic Acid 3

### Nitrilo Trismethylene Phosphonic Acid

Trade name: “Chelest PH-320”, made by Chelest Corporation (active constituent 50 mass %)

### Polyacrylic Acid Polymer

### Isoamylene/Maleic Anhydride Copolymer

Trade name: “Quinflow 542”, made by Zeon Corporation (active constituent 40 mass %)

### Component C

### Nonionic Surfactant 1

### Polyoxyethylene Monostearate

Trade name: “Emalex 6300 M-ST”, made by Nihon-Emulsion Co., Ltd. (active constituent 100 mass %)
Nonionic surfactant 2

### Polyoxyethylene Oleyl Ethoxylate

Trade name: “Nonion E-230”, made by NOF Corporation (active constituent 100 mass %)

### Nonionic Surfactant 3

### Polyoxyethylene Styrenated Phenyl Ether

Trade name: “Noigen EA-207D”, made by Dai-ichi Kogyo Seiyaku Co., Ltd. (active constituent 55 mass %)

### Component D

### Water-Soluble Solvent 1

### Propylene Glycol

Trade name: “Adeka propylene glycol (PG)”, made by Adeka Corporation

### Water-Soluble Solvent 2

### Hexylene Glycol

Trade name: “Hexylene glycol”, made by Dow Chemical Company

### Water-Soluble Solvent 3

### Diethylene Glycol Monobutyl Ether

Trade name: “Butycenol 20”, made by Kyowa Hakko Chemical Co., Ltd.

### Water-Soluble Solvent 4

### Monoethanolamine

Trade name: “Monoethanolamine (MEA)”, made by Nippon Shokubai Co., Ltd.

### Component E

### Cationic Surfactant 1

Alkyl dimethyl hydroxyethyl ammonium salt: C₁₂-to-C₁₄-alkyl dimethyl hydroxyethyl ammonium chloride
Trade name: “Praepagen HY” (made by Clariant Japan, active constituent 40 mass %)

### Cationic Surfactant 2

Benzalkonium chloride: C₁₂-to-C₁₄-alkyl dimethyl benzyl ammonium chloride
Trade name: “Cation G-50” (made by Sanyo Chemical Industries, Ltd. active constituent 50 mass %)

### Cationic Surfactant 3

### Polyhexamethylene Biguanide Hydrochloride:

Poly (n=12)Hexamethylene Biguanide Hydrochloride
Trade name: “Proxel 15” (made by Arch Chemicals, Inc., active constituent 20 mass %)

### Cationic Surfactant 4

Didecyl dimethyl Ammonium Chloride
Trade name: “Bardac-2280” (made by Lonza Japan, active constituent 80 mass %)

### Cationic Surfactant 5

### Didecyl Dimethyl Ammonium Adipate

Trade name: “Osmorin DA-50” (made by Sanyo Chemical Industries, Ltd., active constituent 48 mass %)

### Cationic Surfactant 6

### Hexadecyl Tributyl Phosphonium

Trade name: “KN-1050” (made by KI Chemical Industry Co., Ltd., active constituent 50 mass %)

### Component F

### Anionic Surfactant 1

### Polyoxyethylene Lauryl Ether Phosphoric Acid

Trade name: “Phosphanol ML-220”, made by Toho Chemical Industry Co., Ltd. (active constituent 100 mass %)

### Anionic Surfactant 2

### Polyoxyethylene Alkyl Ether Phosphoric Acid

Trade name: “Phosphanol RA-600”, made by Toho Chemical Industry Co., Ltd. (active constituent 100 mass %)

### Anionic Surfactant 3

### Polyoxyethylene Lauryl Ether Phosphoric Acid

Trade name: “Phosphanol ML-240”, made by Toho Chemical Industry Co., Ltd. (active constituent 100 mass %)
Anionic surfactant 4

### Lauroyl Sarcosine

Trade name: “Sarcosinate LH”, made by Nikko Chemicals Co., Ltd. (active constituent 100 mass %)

### Anionic Surfactant 5

### Polyoxyethylene Lauryl Ether Acetic Acid

Trade name: “BeauliteLCA-25NH”, made by Sanyo Chemical Industries, Ltd. (active constituent 96 mass %)

### Optional Components

### Detergent Builder 1

### Ethylenediaminetetraacetic Acid

Trade name: “KUREWATTO TAA”, made by Nagase Chemtex

### Detergent Builder 2

### Trisodium Citrate Dihydrate

Trade name: “Sodium citrate”, made by Fuso Chemical Co., Ltd.

TABLE 1

| | | Example | | | | |
|---|---|---|---|---|---|---|
| | | 1 | 2 | 3 | 4 | 5 |
| A | Phosphonic acid 1 | | | | | |
| | Phosphonic acid 2 | 0.4 | 2.0 | 4.0 | 8.0 | 20.0 |
| | Phosphonic acid 3 | | | | | |
| | Polycarboxylic | | | | | |
| | acid polymer | | | | | |
| B | Water | Remainder | Remainder | Remainder | Remainder | Remainder |
| Total | | 100.0 | 100.0 | 100.0 | 100.0 | 100.0 |
| Test results | Lubricity 1 | ◯ | ◯ | ◯ | ◯ | ◯ |
| | Deposit | ◯ | ◯ | ◯ | ◯ | ◯ |
| | suppressing | | | | | |
| | ability | | | | | |
| | Detergency | ◯ | ◯ | ◯ | ◯ | ◯ |
| | Scale suppressing | ◯ | ◯ | ◯ | ◯ | ◯ |
| | ability | | | | | |
| | Storage stability | ◯ | ◯ | ◯ | ◯ | ◯ |
| | Stress crack | A | A | A | A | A |
| | preventing ability | | | | | |

TABLE 2

| | | Example | | | | |
|---|---|---|---|---|---|---|
| | | 6 | 7 | 8 | 9 | 10 |
| A | Phosphonic acid 1 | | | 10.0 | 10.0 | 10.0 |
| | Phosphonic acid 2 | 30.0 | 40.0 | 20.0 | 10.0 | 10.0 |
| | Phosphonic acid 3 | | | | | 5.0 |
| | Polycarboxylic | | | | 5.0 | 5.0 |
| | acid polymer | | | | | |
| B | Water | Remainder | Remainder | Remainder | Remainder | Remainder |
| Total | | 100.0 | 100.0 | 100.0 | 100.0 | 100.0 |
| Test results | Lubricity 1 | ◯ | ◯ | ◯ | ◯ | ◯ |
| | Deposit | ◯ | ◯ | ◯ | ◯ | ◯ |
| | suppressing | | | | | |
| | ability | | | | | |
| | Detergency | ◯ | ◯ | ◯ | ◯ | ◯ |
| | Scale suppressing | ◯ | ◯ | ◯ | ◯ | ◯ |
| | ability | | | | | |
| | Storage stability | ◯ | ◯ | ◯ | ◯ | ◯ |
| | Stress crack | A | A | A | A | A |
| | preventing ability | | | | | |

TABLE 3

| | | Example | | | | |
|---|---|---|---|---|---|---|
| | | 11 | 12 | 13 | 14 | 15 |
| A | Phosphonic acid 1 | | 2.0 | | 2.0 | |
| | Phosphonic acid 2 | 4.0 | | 4.0 | | 4.0 |
| | Phosphonic acid 3 | | 1.6 | | 1.6 | |
| | Polycarboxylic | | | | | |
| | acid polymer | | | | | |
| B | Water | Remainder | Remainder | Remainder | Remainder | Remainder |
| D | Water-soluble | 10.0 | 6.0 | | | |
| | solvent 1 | | | | | |
| | Water-soluble | | | | | |
| | solvent 2 | | | | | |
| | Water-soluble | | | | | |
| | solvent 3 | | | | | |
| | Water-soluble | | | | | |
| | solvent 4 | | | | | |
| E | Cationic | | | | | |
| | surfactant 1 | | | | | |
| | Cationic | | | | | |
| | surfactant 2 | | | | | |
| | Cationic | | | | | |
| | surfactant 3 | | | | | |
| | Cationic | | | | | 3.0 |
| | surfactant 4 | | | | | |
| | Cationic | | | | | |
| | surfactant 5 | | | | | |
| | Cationic | | | | | |
| | surfactant 6 | | | | | |
| F | Anionic surfactant 1 | | | | | |
| | Anionic surfactant 2 | | | | 2.0 | |
| | Anionic surfactant 3 | | | 5.0 | | |
| Total | | 100.0 | 100.0 | 100.0 | 100.0 | 100.0 |
| Test results | Lubricity 1 | ◯ | ◯ | ◯ | ◯ | ◯ |
| | Deposit | ◯ | ◯ | ◯ | ◯ | ◯ |
| | suppressing | | | | | |
| | ability | | | | | |
| | Detergency | ◯ | ◯ | ◯ | ◯ | ◯ |
| | Scale suppressing | ◯ | ◯ | Δ | Δ | ◯ |
| | ability | | | | | |
| | Storage stability | ◯ | ◯ | ◯ | ◯ | ◯ |
| | Stress crack | A | A | A | A | A |
| | preventing ability | | | | | |
| | Sterilizing | — | — | — | — | ◯ |
| | ability | | | | | |

TABLE 4

| | | Example |
|---|---|---|
| | | 16 |
| A | Phosphonic acid 1 | 2.0 |
| | Phosphonic acid 2 | |
| | Phosphonic acid 3 | 1.6 |
| | Polycarboxylic | |
| | acid polymer | |
| B | Water | Remainder |
| D | Water-soluble | |
| | solvent 1 | |
| | Water-soluble | |
| | solvent 2 | |
| | Water-soluble | |
| | solvent 3 | |
| | Water-soluble | |
| | solvent 4 | |
| E | Cationic | |
| | surfactant 1 | |
| | Cationic | |
| | surfactant 2 | |
| | Cationic | 6.0 |
| | surfactant 3 | |
| | Cationic | 5.0 |
| | surfactant 4 | |
| | Cationic | |
| | surfactant 5 | |
| | Cationic | |
| | surfactant 6 | |
| F | Anionic surfactant 1 | |
| | Anionic surfactant 2 | |
| | Anionic surfactant 3 | |
| Total | | 100.0 |
| Test results | Lubricity 1 | ◯ |
| | Deposit | ◯ |
| | suppressing | |
| | ability | |
| | Detergency | ◯ |
| | Scale suppressing | ◯ |
| | ability | |
| | Storage stability | ◯ |
| | Stress crack | A |
| | preventing ability | |
| | Sterilizing | ◯ |
| | ability | |

TABLE 5

| | | Example | | | | |
|---|---|---|---|---|---|---|
| | | 17 | 18 | 19 | 20 | 21 |
| A | Phosphonic acid 1 | | | 2.0 | | 5.0 |
| | Phosphonic acid 2 | 2.0 | | | 4.0 | 5.0 |
| | Phosphonic acid 3 | | 4.0 | 1.2 | 4.0 | 8.0 |
| | Polycarboxylic | | | 0.5 | | |
| | acid polymer | | | | | |
| B | Water | Remainder | Remainder | Remainder | Remainder | Remainder |
| C | Nonionic | 0.2 | | 2.0 | 5.0 | 20.0 |
| | surfactant 1 | | | | | |
| | Nonionic | | 3.0 | 2.0 | 5.0 | |
| | surfactant 2 | | | | | |
| | Nonionic | | | 4.0 | | |
| | surfactant 3 | | | | | |
| D | Water-soluble | | | | | |
| | solvent 1 | | | | | |
| | Water-soluble | | | | | |
| | solvent 2 | | | | | |
| | Water-soluble | | | | | |
| | solvent 3 | | | | | |
| | Water-soluble | | | 9.0 | 11.0 | 19.0 |
| | solvent 4 | | | | | |
| Op. | Detergent builder 1 | | | 8.0 | 8.0 | 8.0 |
| | Detergent builder 2 | | | | | |
| Total | | 100.0 | 100.0 | 100.0 | 100.0 | 100.0 |
| A:C weight ratio | | 5:1 | 2:3 | 10:31 | 2:5 | 19:40 |
| A + C active | | 1.2 | 5.0 | 8.2 | 14.0 | 29.5 |
| constituents (mass %) | | | | | | |
| Test results | Lubricity 1 | ◯ | ◯ | ◯ | ◯ | ◯ |
| | Lubricity 2 | ◯ | ◯ | ◯ | ◯ | ◯ |
| | Lubricity 3 | ◯ | ◯ | ◯ | ◯ | ◯ |
| | Deposit | ◯ | ◯ | ◯ | ◯ | ◯ |
| | suppressing | | | | | |
| | ability | | | | | |
| | Detergency | Δ | ◯ | ◯ | ◯ | ◯ |
| | Scale suppressing | ◯ | ◯ | ◯ | ◯ | ◯ |
| | ability | | | | | |
| | Storage stability | ◯ | ◯ | ◯ | ◯ | ◯ |
| | Stress crack | A | A | A | A | A |
| | preventing ability | | | | | |

TABLE 6

| | | Example | | | | |
|---|---|---|---|---|---|---|
| | | 22 | 23 | 24 | 25 | 26 |
| A | Phosphonic acid 1 | | | 2.0 | | 5.0 |
| | Phosphonic acid 2 | | | | 4.0 | 5.0 |
| | Phosphonic acid 3 | | 4.0 | 1.2 | 4.0 | 8.0 |
| | Polycarboxylic | 2.5 | | 0.5 | | |
| | acid polymer | | | | | |
| B | Water | Remainder | Remainder | Remainder | Remainder | Remainder |
| C | Nonionic | 0.2 | | 2.0 | 5.0 | 20.0 |
| | surfactant 1 | | | | | |
| | Nonionic | | 3.0 | 2.0 | 5.0 | |
| | surfactant 2 | | | | | |
| | Nonionic | | | 4.0 | | |
| | surfactant 3 | | | | | |
| D | Water-soluble | 0.8 | 5.0 | | 14.0 | 20.0 |
| | solvent 1 | | | | | |
| | Water-soluble | | | 3.0 | | |
| | solvent 2 | | | | | |
| | Water-soluble | | 4.0 | | | |
| | solvent 3 | | | | | |
| | Water-soluble | | | 9.0 | 11.0 | 19.0 |
| | solvent 4 | | | | | |
| Op. | Detergent builder 1 | | | 8.0 | 8.0 | 8.0 |
| | Detergent builder 2 | | | | | |
| Total | | 100.0 | 100.0 | 100.0 | 100.0 | 100.0 |
| A:C weight ratio | | 5:1 | 2:3 | 10:31 | 2:5 | 19:40 |
| A + C active | | 1.2 | 5.0 | 8.2 | 14.0 | 29.5 |
| constituents (mass %) | | | | | | |
| Test results | Lubricity 1 | ◯ | ◯ | ◯ | ◯ | ◯ |
| | Lubricity 2 | ◯ | ◯ | ◯ | ◯ | ◯ |
| | Lubricity 3 | ◯ | ◯ | ◯ | ◯ | ◯ |
| | Deposit | ◯ | ◯ | ◯ | ◯ | ◯ |
| | suppressing | | | | | |
| | ability | | | | | |
| | Detergency | Δ | ◯ | ◯ | ◯ | ◯ |
| | Scale suppressing | ◯ | ◯ | ◯ | ◯ | ◯ |
| | ability | | | | | |
| | Storage stability | ◯ | ◯ | ◯ | ◯ | ◯ |
| | Stress crack | A | A | A | A | A |
| | preventing ability | | | | | |

TABLE 7

| | | Example | | | | |
|---|---|---|---|---|---|---|
| | | 27 | 28 | 29 | 30 | 31 |
| A | Phosphonic acid 1 | 2.0 | | 5.0 | 2.0 | |
| | Phosphonic acid 2 | | 4.0 | 5.0 | | 4.0 |
| | Phosphonic acid 3 | 1.2 | 4.0 | 8.0 | 1.2 | 4.0 |
| | Polycarboxylic | 0.5 | | | 0.5 | |
| | acid polymer | | | | | |
| B | Water | Remainder | Remainder | Remainder | Remainder | Remainder |
| C | Nonionic | 2.0 | | 20.0 | 2.0 | |
| | surfactant 1 | | | | | |
| | Nonionic | | 5.0 | | | 5.0 |
| | surfactant 2 | | | | | |
| | Nonionic | 3.0 | | | 3.0 | |
| | surfactant 3 | | | | | |
| D | Water-soluble | | | | | 14.0 |
| | solvent 1 | | | | | |
| | Water-soluble | | | | 3.0 | |
| | solvent 2 | | | | | |
| | Water-soluble | | | | | |
| | solvent 3 | | | | | |
| | Water-soluble | 9.0 | 11.0 | 19.0 | 9.0 | 11.0 |
| | solvent 4 | | | | | |
| E | Cationic | | | | | |
| | surfactant 1 | | | | | |
| | Cationic | | | | | |
| | surfactant 2 | | | | | |
| | Cationic | | | | | |
| | surfactant 3 | | | | | |
| | Cationic | | | | | |
| | surfactant 4 | | | | | |
| | Cationic | | | | | |
| | surfactant 5 | | | | | |
| | Cationic | | | | | |
| | surfactant 6 | | | | | |
| F | Anionic surfactant 1 | | | 5.0 | | |
| | Anionic surfactant 2 | 2.0 | | 5.0 | 2.0 | |
| | Anionic surfactant 3 | | 5.0 | | | 5.0 |
| Op. | Detergent builder 1 | 8.0 | 8.0 | 8.0 | 8.0 | 8.0 |
| | Detergent builder 2 | | | | | |
| Total | | 100.0 | 100.0 | 100.0 | 100.0 | 100.0 |
| A:C weight ratio | | 5:14 | 2:5 | 19:40 | 5:14 | 2:5 |
| A + C active | | 7.7 | 14.0 | 29.5 | 7.7 | 14.0 |
| constituents (mass %) | | | | | | |
| Test results | Lubricity 1 | ◯ | ◯ | ◯ | ◯ | ◯ |
| | Lubricity 2 | ◯ | ◯ | ◯ | ◯ | ◯ |
| | Lubricity 3 | ◯ | ◯ | ◯ | ◯ | ◯ |
| | Deposit | ◯ | ◯ | ◯ | ◯ | ◯ |
| | suppressing | | | | | |
| | ability | | | | | |
| | Detergency | ◯ | ◯ | ◯ | ◯ | ◯ |
| | Scale suppressing | Δ | Δ | Δ | Δ | Δ |
| | ability | | | | | |
| | Storage stability | ◯ | ◯ | ◯ | ◯ | ◯ |
| | Stress crack | A | A | A | A | A |
| | preventing ability | | | | | |

TABLE 8

| | | Example |
|---|---|---|
| | | 32 |
| A | Phosphonic acid 1 | 5.0 |
| | Phosphonic acid 2 | 4.0 |
| | Phosphonic acid 3 | 8.0 |
| | Polycarboxylic | |
| | acid polymer | |
| B | Water | Remainder |
| C | Nonionic | 10.0 |
| | surfactant 1 | |
| | Nonionic | |
| | surfactant 2 | |
| | Nonionic | |
| | surfactant 3 | |
| D | Water-soluble | 20.0 |
| | solvent 1 | |
| | Water-soluble | |
| | solvent 2 | |
| | Water-soluble | |
| | solvent 3 | |
| | Water-soluble | 19.0 |
| | solvent 4 | |
| E | Cationic | |
| | surfactant 1 | |
| | Cationic | |
| | surfactant 2 | |
| | Cationic | |
| | surfactant 3 | |
| | Cationic | |
| | surfactant 4 | |
| | Cationic | |
| | surfactant 5 | |
| | Cationic | |
| | surfactant 6 | |
| F | Anionic surfactant 1 | 5.0 |
| | Anionic surfactant 2 | 5.0 |
| | Anionic surfactant 3 | |
| Op. | Detergent builder 1 | 8.0 |
| | Detergent builder 2 | |
| Total | | 100.0 |
| A:C weight ratio | | 9:10 |
| A + C active | | 19.0 |
| constituents (mass %) | | |
| Test results | Lubricity 1 | ◯ |
| | Lubricity 2 | ◯ |
| | Lubricity 3 | ◯ |
| | Deposit | ◯ |
| | suppressing | |
| | ability | |
| | Detergency | ◯ |
| | Scale suppressing | Δ |
| | ability | |
| | Storage stability | ◯ |
| | Stress crack | A |
| | preventing ability | |
| | Sterilizing | |
| | ability | |

TABLE 9

| | | Example | | | | |
|---|---|---|---|---|---|---|
| | | 33 | 34 | 35 | 36 | 37 |
| A | Phosphonic acid 1 | | | 2.0 | | 5.0 |
| | Phosphonic acid 2 | | | | 4.0 | 5.0 |
| | Phosphonic acid 3 | | 4.0 | 1.2 | 4.0 | 8.0 |
| | Polycarboxylic | 2.5 | | 0.5 | | |
| | acid polymer | | | | | |
| B | Water | Remainder | Remainder | Remainder | Remainder | Remainder |
| C | Nonionic | 0.2 | | 2.0 | 5.0 | 20.0 |
| | surfactant 1 | | | | | |
| | Nonionic | | 3.0 | 2.0 | 5.0 | |
| | surfactant 2 | | | | | |
| | Nonionic | | | 4.0 | | |
| | surfactant 3 | | | | | |
| D | Water-soluble | 0.8 | 5.0 | | 14.0 | 20.0 |
| | solvent 1 | | | | | |
| | Water-soluble | | | 3.0 | | |
| | solvent 2 | | | | | |
| | Water-soluble | | 4.0 | | | |
| | solvent 3 | | | | | |
| | Water-soluble | | | 9.0 | 11.0 | 19.0 |
| | solvent 4 | | | | | |
| E | Cationic | 22.5 | | | 10.0 | |
| | surfactant 1 | | | | | |
| | Cationic | 30.0 | 10.0 | | | |
| | surfactant 2 | | | | | |
| | Cationic | 30.0 | | 6.0 | | |
| | surfactant 3 | | | | | |
| | Cationic | | | 5.0 | | 5.0 |
| | surfactant 4 | | | | | |
| | Cationic | | 5.0 | | 5.0 | |
| | surfactant 5 | | | | | |
| | Cationic | | | | | |
| | surfactant 6 | | | | | |
| Op. | Detergent builder 1 | | | 8.0 | 8.0 | 8.0 |
| | Detergent builder 2 | | | | | |
| Total | | 100.0 | 100.0 | 100.0 | 100.0 | 100.0 |
| A:C weight ratio | | 5:1 | 2:3 | 10:31 | 2:5 | 19:40 |
| A + C active | | 1.2 | 5.0 | 8.2 | 14.0 | 29.5 |
| constituents (mass %) | | | | | | |
| Test results | Lubricity 1 | ◯ | ◯ | ◯ | ◯ | ◯ |
| | Lubricity 2 | ◯ | ◯ | ◯ | ◯ | ◯ |
| | Lubricity 3 | ◯ | ◯ | ◯ | ◯ | ◯ |
| | Deposit | ◯ | ◯ | ◯ | ◯ | ◯ |
| | suppressing | | | | | |
| | ability | | | | | |
| | Detergency | Δ | ◯ | ◯ | ◯ | ◯ |
| | Scale suppressing | ◯ | ◯ | ◯ | ◯ | ◯ |
| | ability | | | | | |
| | Storage stability | ◯ | ◯ | ◯ | ◯ | ◯ |
| | Stress crack | A | A | A | A | A |
| | preventing ability | | | | | |
| | Sterilizing | ◯ | ◯ | ◯ | ◯ | ◯ |
| | ability | | | | | |

TABLE 10

| | | Example | | | | |
|---|---|---|---|---|---|---|
| | | 38 | 39 | 40 | 41 | 42 |
| A | Phosphonic acid 1 | | 1.0 | | | 10.0 |
| | Phosphonic acid 2 | | | | 8.0 | |
| | Phosphonic acid 3 | | | 5.0 | | 8.0 |
| | Polycarboxylic | 0.5 | 1.0 | | 5.0 | |
| | acid polymer | | | | | |
| B | Water | Remainder | Remainder | Remainder | Remainder | Remainder |
| C | Nonionic | | 2.0 | 1.5 | | 0.5 |
| | surfactant 1 | | | | | |
| | Nonionic | 2.0 | | 1.0 | | 0.5 |
| | surfactant 2 | | | | | |
| | Nonionic | | | | 4.0 | |
| | surfactant 3 | | | | | |
| D | Water-soluble | 5.0 | | | | |
| | solvent 1 | | | | | |
| | Water-soluble | | 5.0 | | | |
| | solvent 2 | | | | | |
| | Water-soluble | | | | | 5.0 |
| | solvent 3 | | | | | |
| | Water-soluble | | | 9.0 | | 19.0 |
| | solvent 4 | | | | | |
| E | Cationic | | | 3.0 | | 3.0 |
| | surfactant 1 | | | | | |
| | Cationic | | 3.0 | | 3.0 | |
| | surfactant 2 | | | | | |
| | Cationic | 6.0 | | 3.0 | | 6.0 |
| | surfactant 3 | | | | | |
| | Cationic | | | | 3.0 | |
| | surfactant 4 | | | | | |
| | Cationic | | 3.0 | | | |
| | surfactant 5 | | | | | |
| | Cationic | | | | | |
| | surfactant 6 | | | | | |
| Op. | Detergent builder 1 | | | 8.0 | | 8.0 |
| | Detergent builder 2 | 5.0 | 1.0 | | | |
| Total | | 100.0 | 100.0 | 100.0 | 100.0 | 100.0 |
| A:C weight ratio | | 1:10 | 1:2 | 1:1 | 30:11 | 10:1 |
| A + C active | | 2.2 | 3.0 | 5.0 | 8.2 | 11.0 |
| constituents (mass %) | | | | | | |
| Test results | Lubricity 1 | ◯ | ◯ | ◯ | ◯ | ◯ |
| | Lubricity 2 | ◯ | ◯ | ◯ | ◯ | ◯ |
| | Lubricity 3 | ◯ | ◯ | ◯ | ◯ | ◯ |
| | Deposit | ◯ | ◯ | ◯ | ◯ | ◯ |
| | suppressing | | | | | |
| | ability | | | | | |
| | Detergency | ◯ | ◯ | ◯ | ◯ | ◯ |
| | Scale suppressing | ◯ | ◯ | ◯ | ◯ | ◯ |
| | ability | | | | | |
| | Storage stability | ◯ | ◯ | ◯ | ◯ | ◯ |
| | Stress crack | A | A | A | A | A |
| | preventing ability | | | | | |
| | Sterilizing | ◯ | ◯ | ◯ | ◯ | ◯ |
| | ability | | | | | |

TABLE 11

| | | Example | | | | |
|---|---|---|---|---|---|---|
| | | 43 | 44 | 45 | 46 | 47 |
| A | Phosphonic acid 1 | 6.6 | | | 2.0 | |
| | Phosphonic acid 2 | 10.0 | 2.0 | | | 4.0 |
| | Phosphonic acid 3 | 22.0 | | | 1.6 | 4.0 |
| | Polycarboxylic | | | 5.0 | | |
| | acid polymer | | | | | |
| B | Water | Remainder | Remainder | Remainder | Remainder | Remainder |
| C | Nonionic | 0.5 | 0.2 | 2.0 | 2.0 | 5.0 |
| | surfactant 1 | | | | | |
| | Nonionic | 0.5 | | 3.0 | 2.0 | 5.0 |
| | surfactant 2 | | | | | |
| | Nonionic | | | | 4.0 | |
| | surfactant 3 | | | | | |
| D | Water-soluble | 5.0 | 0.8 | 5.0 | 6.0 | 20.0 |
| | solvent 1 | | | | | |
| | Water-soluble | | | | | |
| | solvent 2 | | | | | |
| | Water-soluble | | | 5.0 | | |
| | solvent 3 | | | | | |
| | Water-soluble | 28.5 | | | 9.5 | 11.5 |
| | solvent 4 | | | | | |
| E | Cationic | | | | | 10.0 |
| | surfactant 1 | | | | | |
| | Cationic | | 5.0 | 5.0 | | |
| | surfactant 2 | | | | | |
| | Cationic | | | | 6.0 | |
| | surfactant 3 | | | | | |
| | Cationic | 3.0 | | | 5.0 | |
| | surfactant 4 | | | | | |
| | Cationic | 3.0 | | | | 5.0 |
| | surfactant 5 | | | | | |
| | Cationic | | | | | |
| | surfactant 6 | | | | | |
| Op. | Detergent builder 1 | 8.0 | | | 8.0 | 8.0 |
| | Detergent builder 2 | | 3.0 | 5.0 | | |
| Total | | 100.0 | 100.0 | 100.0 | 100.0 | 100.0 |
| A:C weight ratio | | 20:1 | 5:1 | 2:5 | 10:31 | 2:5 |
| A + C active | | 21.0 | 1.2 | 7.0 | 8.2 | 14.0 |
| constituents (mass %) | | | | | | |
| Test results | Lubricity 1 | ◯ | ◯ | ◯ | ◯ | ◯ |
| | Lubricity 2 | ◯ | ◯ | ◯ | ◯ | ◯ |
| | Lubricity 3 | ◯ | ◯ | ◯ | ◯ | ◯ |
| | Deposit | ◯ | ◯ | ◯ | ◯ | ◯ |
| | suppressing | | | | | |
| | ability | | | | | |
| | Detergency | ◯ | ◯ | ◯ | ◯ | ◯ |
| | Scale suppressing | ◯ | ◯ | ◯ | ◯ | ◯ |
| | ability | | | | | |
| | Storage stability | ◯ | ◯ | ◯ | ◯ | ◯ |
| | Stress crack | A | A | A | A | A |
| | preventing ability | | | | | |
| | Sterilizing | ◯ | ◯ | ◯ | ◯ | ◯ |
| | ability | | | | | |

TABLE 12

| | | Example | | | | |
|---|---|---|---|---|---|---|
| | | 48 | 49 | 50 | 51 | 52 |
| A | Phosphonic acid 1 | | 2.0 | | | 5.0 |
| | Phosphonic acid 2 | 4.0 | | 4.0 | 4.0 | 4.0 |
| | Phosphonic acid 3 | | 1.6 | 4.0 | | 8.0 |
| | Polycarboxylic | | | | | |
| | acid polymer | | | | | |
| B | Water | Remainder | Remainder | Remainder | Remainder | Remainder |
| C | Nonionic | 2.5 | 2.0 | | | 10.0 |
| | surfactant 1 | | | | | |
| | Nonionic | 3.0 | | 5.0 | 3.0 | |
| | surfactant 2 | | | | | |
| | Nonionic | | 4.0 | | | |
| | surfactant 3 | | | | | |
| D | Water-soluble | 10.0 | 6.0 | 20.0 | 10.0 | 20.0 |
| | solvent 1 | | | | | |
| | Water-soluble | | | | | |
| | solvent 2 | | | | | |
| | Water-soluble | | | | | |
| | solvent 3 | | | | | |
| | Water-soluble | 7.5 | 9.5 | 11.5 | 7.5 | 19.0 |
| | solvent 4 | | | | | |
| E | Cationic | | | 10.0 | | |
| | surfactant 1 | | | | | |
| | Cationic | | | | | |
| | surfactant 2 | | | | | |
| | Cationic | | 6.0 | | | |
| | surfactant 3 | | | | | |
| | Cationic | 3.0 | 5.0 | | 3.0 | 2.5 |
| | surfactant 4 | | | | | |
| | Cationic | | | 5.0 | | |
| | surfactant 5 | | | | | |
| | Cationic | | | | | |
| | surfactant 6 | | | | | |
| F | Anionic surfactant 1 | | | | 1.0 | 5.0 |
| | Anionic surfactant 2 | | 2.0 | | | 5.0 |
| | Anionic surfactant 3 | | | 5.0 | 1.5 | |
| Op. | Detergent builder 1 | 8.2 | 8.0 | 8.0 | 8.2 | 8.0 |
| | Detergent builder 2 | | | | | |
| Total | | 100.0 | 100.0 | 100.0 | 100.0 | 100.0 |
| A:C weight ratio | | 4:11 | 10:21 | 4:5 | 2:3 | 9:10 |
| A + C active | | 7.5 | 6.2 | 9.0 | 5.0 | 19.0 |
| constituents (mass %) | | | | | | |
| Test results | Lubricity 1 | ◯ | ◯ | ◯ | ◯ | ◯ |
| | Lubricity 2 | ◯ | ◯ | ◯ | ◯ | ◯ |
| | Lubricity 3 | ◯ | ◯ | ◯ | ◯ | ◯ |
| | Deposit | ◯ | ◯ | ◯ | ◯ | ◯ |
| | suppressing | | | | | |
| | ability | | | | | |
| | Detergency | ◯ | ◯ | ◯ | ◯ | ◯ |
| | Scale suppressing | ◯ | Δ | Δ | Δ | Δ |
| | ability | | | | | |
| | Storage stability | ◯ | ◯ | ◯ | ◯ | ◯ |
| | Stress crack | A | A | A | A | A |
| | preventing ability | | | | | |
| | Sterilizing | ◯ | ◯ | ◯ | ◯ | ◯ |
| | ability | | | | | |

TABLE 13

| | | Example | | | |
|---|---|---|---|---|---|
| | | 53 | 54 | 55 | 56 |
| A | Phosphonic acid 1 | | | | 2.0 |
| | Phosphonic acid 2 | 4.0 | 4.0 | 4.0 | |
| | Phosphonic acid 3 | | | | 1.6 |
| | Polycarboxylic | | | | |
| | acid polymer | | | | |
| B | Water | Remainder | Remainder | Remainder | Remainder |
| C | Nonionic | | | 2.5 | 2.0 |
| | surfactant 1 | | | | |
| | Nonionic | 3.0 | 3.0 | 3.0 | |
| | surfactant 2 | | | | |
| | Nonionic | | | | 4.0 |
| | surfactant 3 | | | | |
| D | Water-soluble | 10.0 | 10.0 | | |
| | solvent 1 | | | | |
| | Water-soluble | | | | |
| | solvent 2 | | | | |
| | Water-soluble | | | | |
| | solvent 3 | | | | |
| | Water-soluble | 7.5 | 7.5 | | |
| | solvent 4 | | | | |
| E | Cationic | | | | |
| | surfactant 1 | | | | |
| | Cationic | | | | |
| | surfactant 2 | | | | |
| | Cationic | | | | 6.0 |
| | surfactant 3 | | | | |
| | Cationic | 3.0 | 3.0 | 3.0 | 5.0 |
| | surfactant 4 | | | | |
| | Cationic | | | | |
| | surfactant 5 | | | | |
| | Cationic | | | | |
| | surfactant 6 | | | | |
| F | Anionic | 2.5 | | | |
| | surfactant 4 | | | | |
| | Anionic | | 2.5 | | |
| | surfactant 5 | | | | |
| Op. | Detergent | 8.2 | 8.2 | | |
| | builder 1 | | | | |
| | Detergent | | | | |
| | builder 2 | | | | |
| Total | | 100.0 | 100.0 | 100.0 | 100.0 |
| A:C weight ratio | | 2:3 | 2:3 | 4:11 | 10:21 |
| A + C active | | 5.0 | 5.0 | 7.5 | 6.2 |
| constituents (mass %) | | | | | |
| Test | Lubricity 1 | ◯ | ◯ | ◯ | ◯ |
| re- | Lubricity 2 | ◯ | ◯ | ◯ | ◯ |
| sults | Lubricity 3 | ◯ | ◯ | ◯ | ◯ |
| | Deposit | ◯ | ◯ | ◯ | ◯ |
| | suppressing | | | | |
| | ability | | | | |
| | Detergency | ◯ | ◯ | ◯ | ◯ |
| | Scale suppressing | Δ | Δ | ◯ | ◯ |
| | ability | | | | |
| | Storage stability | ◯ | ◯ | ◯ | ◯ |
| | Stress crack | A | A | A | A |
| | preventing ability | | | | |
| | Sterilizing | ◯ | ◯ | ◯ | ◯ |
| | ability | | | | |

TABLE 14

| | | Comparative example | | | | |
|---|---|---|---|---|---|---|
| | | 1 | 2 | 3 | 4 | 5 |
| A | Phosphonic acid 1 | | | | | |
| | Phosphonic acid 2 | | | | | |
| | Phosphonic acid 3 | | | | | |
| | Polycarboxylic | | | | | |
| | acid polymer | | | | | |
| B | Water | Remainder | Remainder | Remainder | Remainder | Remainder |
| C | Nonionic | 5.0 | | 2.0 | | |
| | surfactant 1 | | | | | |
| | Nonionic | | | 6.0 | | |
| | surfactant 2 | | | | | |
| | Nonionic | | 10.0 | | | |
| | surfactant 3 | | | | | |
| D | Water-soluble | | | | | |
| | solvent 1 | | | | | |
| | Water-soluble | | | 5.0 | | |
| | solvent 2 | | | | | |
| | Water-soluble | | | | | |
| | solvent 3 | | | | | |
| | Water-soluble | | 45.0 | | | |
| | solvent 4 | | | | | |
| E | Cationic | | | | | |
| | surfactant 1 | | | | | |
| | Cationic | | | | | |
| | surfactant 2 | | | | | |
| | Cationic | | | | | |
| | surfactant 3 | | | | | |
| | Cationic | | | 4.0 | | 5.0 |
| | surfactant 4 | | | | | |
| | Cationic | | | | | |
| | surfactant 5 | | | | | |
| | Cationic | | | | | |
| | surfactant 6 | | | | | |
| F | Anionic surfactant 1 | | | | | |
| | Anionic surfactant 2 | | | | | |
| | Anionic surfactant 3 | | | | | |
| Op. | Detergent builder 1 | | | | | |
| | Detergent builder 2 | | | | 5.0 | |
| Total | | 100.0 | 100.0 | 100.0 | 100.0 | 100.0 |
| A:C weight ratio | | — | — | — | — | — |
| A + C active | | 5.0 | 10.0 | 8.0 | 0 | 0 |
| constituents (mass %) | | | | | | |
| Test results | Lubricity 1 | X | X | X | X | X |
| | Lubricity 2 | ◯ | ◯ | ◯ | X | X |
| | Lubricity 3 | ◯ | ◯ | ◯ | X | X |
| | Deposit | ◯ | ◯ | X | X | X |
| | suppressing | | | | | |
| | ability | | | | | |
| | Detergency | ◯ | ◯ | ◯ | X | X |
| | Scale suppressing | ◯ | ◯ | X | ◯ | X |
| | ability | | | | | |
| | Storage stability | ◯ | X | ◯ | ◯ | ◯ |
| | Stress crack | B | B | D | A | C |
| | preventing ability | | | | | |
| | Sterilizing | X | X | ◯ | X | X |
| | ability | | | | | |

TABLE 15

| | | Comparative |
|---|---|---|
| | | example |
| | | 6 |
| A | Phosphonic acid 1 | |
| | Phosphonic acid 2 | |
| | Phosphonic acid 3 | |
| | Polycarboxylic | |
| | acid polymer | |
| B | Water | Remainder |
| C | Nonionic | 2.0 |
| | surfactant 1 | |
| | Nonionic | |
| | surfactant 2 | |
| | Nonionic | |
| | surfactant 3 | |
| D | Water-soluble | |
| | solvent 1 | |
| | Water-soluble | 5.0 |
| | solvent 2 | |
| | Water-soluble | |
| | solvent 3 | |
| | Water-soluble | |
| | solvent 4 | |
| E | Cationic | |
| | surfactant 1 | |
| | Cationic | |
| | surfactant 2 | |
| | Cationic | |
| | surfactant 3 | |
| | Cationic | 4.0 |
| | surfactant 4 | |
| | Cationic | |
| | surfactant 5 | |
| | Cationic | |
| | surfactant 6 | |
| F | Anionic surfactant 1 | |
| | Anionic surfactant 2 | 6.0 |
| | Anionic surfactant 3 | |
| Op. | Detergent builder 1 | |
| | Detergent builder 2 | |
| Total | | 100.0 |
| A:C weight ratio | | — |
| A + C active | | 2.0 |
| constituents (mass %) | | |
| Test results | Lubricity 1 | ◯ |
| | Lubricity 2 | ◯ |
| | Lubricity 3 | ◯ |
| | Deposit | X |
| | suppressing | |
| | ability | |
| | Detergency | X |
| | Scale suppressing | X |
| | ability | |
| | Storage stability | ◯ |
| | Stress crack | C |
| | preventing ability | |
| | Sterilizing | ◯ |
| | ability | |

From the results in Tables 1 to 15, it can seen that all of examples 1 to 56 of the lubricant composition of the present invention have excellent performance for all of the six items lubricity, detergency, storage stability, scale suppressing ability, deposit suppressing ability, and stress crack preventing ability. Moreover, it can be seen that examples 15, 16, and 33 to 56 are lubricant compositions having excellent sterilizing ability, there being no limitation on the type of the cationic surfactant.

Moreover, for the problem (task) that, in a filling process or the like for a beverage such as green tea, black tea, Java tea, coffee, beer or cola, particularly green tea or black tea, upon the beverage overflowing and becoming attached, components of the beverage and components of the conveyor lubricant form a water-insoluble salt (deposit) through chemical reaction, which becomes attached as a brown-black deposit to the conveyor and also to the bottoms and sides of PET containers, it can be seen that for all of examples 1 to 56 of the lubricant composition of the present invention, the deposit suppressing ability is excellent.

Moreover, for lubricity test 1 (stainless steel convevor), upon carrying out the test using containers as in (1) and (2) below as test containers, the value of the coefficient of friction (μ) was less than 0.2, confirming usability in practice. Note that here, examples 49 to 54 were used as the test lubricant compositions. (1) Two 330 ml test glass containers (total weight 1180 g) (2) Two 350 ml test aluminum cans (total weight 750 g)

Similarly, for lubricity test 2 (resin conveyor), upon carrying out the test using containers as in (1) and (2) below as test containers, the value of the coefficient of friction (μ) was less than 0.1, confirming usability in practice. Note that here, examples 18, and 49 to 54 were used as the test lubricant compositions. (1) Two 330 ml test glass containers (total weight 1180 g) (2) Two 350 ml test aluminum cans (total weight 750 g)

1. A bottle conveyor lubricant composition characterized by containing
(A) at least one chelating compound selected from the group consisting of phosphonic acids, polyacrylic acid polymers and/or salts thereof, wherein the chelating compound is present in an amount from about 0.2% to about 20% by weight of the total composition;
(B) water; and
(C) a nonionic surfactant, wherein the nonionic surfactant is present in an amount from about 0.2% to about 20% by weight of the total composition.

2. The bottle conveyor lubricant composition according to claim 1, wherein the salts comprise alkali metal salts, alkaline earth metal salts, ammonium salts, or amine salts.

3. The bottle conveyor lubricant composition according to claim 1, characterized by further containing (D) a water-soluble solvent.

4. The bottle conveyor lubricant composition according to claim 1, characterized by further containing (E) a cationic surfactant.

5. The bottle conveyor lubricant composition according to claim 1, characterized by further containing (F) at least one anionic surfactant selected from the group represented by general formulae (1) to (3): 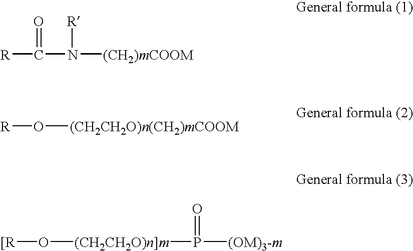
wherein R is a C₃-C₂₀ alkyl group or alkenyl group, R′ is a C₁-C₄ alkyl group, n is an integer from 1 to 8, m is an integer of 1 or 2, and M is hydrogen, an alkali metal, an amine, or an alkanolamine.

6. The bottle conveyor lubricant composition according to claim 1, characterized in that the nonionic surfactant component (C) comprises at least one selected from polyoxyalkylene alkyl ethers, polyoxyalkylene fatty acid esters, block copolymers of polyoxyethylene (EO) and polyoxypropylene (PO), polyoxyethylene oleyl ether (EO), and polyoxyalkylene styrenated phenyl ethers.

7. The bottle conveyor lubricant composition according to claim 3, characterized in that the water-soluble solvent component (D) comprises at least one selected from the group consisting of ethyl alcohol, isopropyl alcohol, propylene glycol, polyethylene glycol, hexylene glycol, ethylene glycol ethers including ethylene glycol monomethyl ether, ethylene glycol monoethyl ether, ethylene glycol monopropyl ether and ethylene glycol monobutyl ether, diethylene glycol ethers including diethylene glycol monomethyl ether, diethylene glycol monoethyl ether, diethylene glycol monopropyl ether and diethylene glycol monobutyl ether, propylene glycol monomethyl ether, dipropylene glycol monomethyl ether, hexylene glycol, isoprene glycol, glycerol, and monoethanolamine.

8. A bottle conveyor lubricant composition characterized in that the bottle conveyor lubricant composition according to claim 1 is used for conveying polyalkylene terephthalate containers.

9. A bottle conveyor lubricant composition characterized in that the bottle conveyor lubricant composition according to claim 1 has an effect of suppressing occurrence of deposit upon contact with a beverage to be filled on a conveyor.

10. A method of use, comprising using the bottle conveyor lubricant composition according to claim 1 for conveying and moving containers on a conveyor by supplying, spraying, or applying onto the conveyor a diluted lubricant liquid obtained by diluting the lubricant composition with water or hot water to a component (A) concentration of 10 to 500 mg/L.

11. A method of use, comprising using the bottle conveyor lubricant composition according to claim 1 for conveying and moving containers by supplying, spraying, or applying onto a conveyor a mixture of a diluted lubricant liquid obtained by diluting the lubricant composition with water or hot water to a component (A) concentration of 10 to 500 mg/L, and at least one selected from sterilizing agent aqueous solutions obtained by diluting, with water or hot water, a hypochlorite, peracetic acid, hydrogen peroxide or iodine to not less than 5 mg/L or chlorine dioxide to not less than 0.05 mg/L.

12. A method of use, comprising using the bottle conveyor lubricant composition according to claim 1 for conveying and moving containers by separately supplying, spraying, or applying onto a conveyor a diluted lubricant liquid obtained by diluting the lubricant composition with water or hot water to a component (A) concentration of 10 to 500 mg/L, and at least one selected from sterilizing agent aqueous solutions obtained by diluting, with water or how water, a hypochlorite, peracetic acid, hydrogen peroxide or iodine to not less than 5 mg/L or chlorine dioxide to not less than 0.05 mg/L.

13. The bottle conveyor lubricant composition according to claim 1, wherein the chelating agent is selected from the group consisting of 1-hydroxyethylidene-1,1-diphosphonic acid, 2-phosphono-1,2,4-butanetricarboxylic acid, nitrilo trismethylene phosphonic acid, and isoamylene/maleic anhydride copolymer.

14. The bottle conveyor lubricant composition according to claim 13, wherein the chelating agent comprises 2-phosphono-1,2,4-butanetricarboxylic acid, ethylene diamine tetra acetic acid, and combinations thereof.

15. The bottle conveyor lubricant composition according to claim 1, wherein the nonionic surfactant is selected from the group consisting of polyoxyethylene monostearate, polyoxyethylene oleyl ethoxylate, and polyoxyethylene styrenated phenyl ether.

16. The bottle conveyor lubricant composition according to claim 4, wherein the cationic surfactant is selected from the group consisting of a alkyl dimethyl hydroxyethyl ammonium salt, an alkyl dimethyl benzyl ammonium salt, a polyhexamethylene biguanide hydrochloride, didecyl dimethyl ammonium chloride, didecyl dimethyl ammonium adipate, and hexadecyl tributyl phosphonium.

17. The bottle conveyor lubricant composition according to claim 5, wherein the anionic surfactant is selected from the group consisting of polyoxyethylene lauryl ether phosphoric acid, polyoxyethylene alkyl ether phosphoric acid, lauroyl sarcosine, and polyoxyethylene lauryl ether acetic acid.

18. The bottle conveyor lubricant composition according to claim 1, further comprising a detergent builder.

19. The bottle conveyor lubricant composition according to claim 18,
wherein the detergent builder comprises ethylenediaminetetraacetic acid, trisodium citrate dehydrate, and combinations thereof.

20. A method for lubricating a bottle conveyor, comprising applying a composition according to claim 1 onto a bottle conveyor.

21. The method according to claim 20, wherein the bottle conveyor comprises stainless steel or resin.

22. The method according to claim 20, wherein the conveyor is used to transport beverage containers.

23. The method of claim 22, wherein the beverage containers are polyethylene terephthalate containers.

24. The method of claim 22, wherein the beverage containers contain a green tea, a black tea, a Java tea, coffee, beer, a cola, a carbonated beverage or a milk beverage.

25. A method for sterilizing a bottle conveyor, comprising applying onto the conveyor a mixture comprising (1) a composition comprising (A) at least one chelating compound selected from the group consisting of phosphonic acids, polyacrylic acid polymers and/or salts thereof, wherein the chelating compound is present in an amount from about 0.2% to about 20% by weight of the total composition, (B) water, (C) a nonionic surfactant, wherein the nonionic surfactant is present in an amount from about 0.2% to about 20% by weight of the total composition, and (2) a sterilizing agent.

26. The method of claim 25, wherein the sterilizing agent comprises water and a hypochlorite, peracetic acid, hydrogen peroxide, chlorine dioxide, or iodine, or a combination thereof.
